(19) United States
(12) Patent Application Publication
(10) Pub. No.: US 2016/0324773 A1
(43) Pub. Date: Nov. 10, 2016

(54) FILM DOSAGE FORMS CONTAINING AMORPHOUS ACTIVE AGENTS

(71) Applicant: IntelGenx Corp., St-Laurent (CA)

(72) Inventors: Nadine Paiement, St-Laurent (CA); Rodolphe Obeid, St-Laurent (CA); Billal Tir, Montreal (CA)

(73) Assignee: IntelGenx Corp., St-Laurent (CA)

(21) Appl. No.: 15/216,903

(22) Filed: Jul. 22, 2016

Related U.S. Application Data
(62) Division of application No. 14/630,699, filed on Feb. 25, 2015.

(51) Int. Cl. A61K 9/00; A61K 31/4985; A61K 47/38; A61K 31/5513

(52) U.S. Cl. CPC A61K 9/0056; A61K 31/5513; A61K 31/4985; A61K 47/38

(57) ABSTRACT

Oral thin film dosage form of a stable dispersion of non-solubilized amorphous or partially amorphous active agent(s), having a mean particle size diameter D50 equal or less than 250 μm, that remains uniformly distributed within a film matrix and contains at least one film forming polymer, and optional pharmaceutically-acceptable excipients, such as diluents, plasticizers, surfactants, sweeteners, and taste-masking agent(s), are prepared by a process including first providing the active agent in an amorphous particle form having a mean particle size diameter D50 equal or less than 250 μm. Next, the active agent is suspended in a liquid film-forming formulation without dissolving the active agent. Therefore, the solvent is removed to form a film.

### CROSS-REFERENCE TO RELATED APPLICATIONS

This application is a division of application Ser. No. 14/630,699, filed Feb. 25, 2015, which is incorporated herein by reference in its entirety.

### FIELD OF THE DISCLOSURE

This disclosure relates to a process for preparing oral film dosage forms, and more particularly to preparation of oral film dosage forms that exhibit enhanced bioavailability.

### BACKGROUND OF THE DISCLOSURE

It is often desirable to reliably increase the bioavailability of a pharmaceutically active agent. Advantages include lower dosage amounts and enhanced efficacy. Although amorphous active agents are often known to achieve enhanced bioavailability as compared with crystalline forms, it is typically very difficult to maintain the amorphous active agents in a stable amorphous state. Rather, amorphous active agents tend to agglomerate and transform into crystalline particles.

It is known that amorphous materials often exhibit higher solubility and a faster dissolution rate, such as in the aqueous liquid medium found in the alimentary tract (e.g., saliva, stomach and intestinal fluids), as compared with the generally thermodynamically favored crystalline forms of the same material. As a consequence, amorphous forms of active agent(s) are expected to exhibit improved bioavailability as compared with crystalline forms of the active agent(s). Numerous techniques, such as extrusion, solvent evaporation, physical mixture, lyophilization, co-precipitation, nanosuspension, melting, co-melting and spray drying, are well known to fully or partially convert materials, including many active agent(s), from their crystalline state to the amorphous state.

Despite the potentially improved bioavailability of active agent(s)s in the amorphous form, crystalline materials are generally preferred in the pharmaceutical industry, because the amorphous forms often exhibit poor thermodynamic stability, greater chemical instability, altered mechanical properties, and greater hygroscopicity. These undesirable properties, if not anticipated, prevented or controlled, can lead to processing and storage difficulties that render the use of amorphous forms of active agent(s)s impracticable due to excessive processing costs, shorter shelf life, formation of undesirable degradation products, or a combination of these problems. Moreover, because the amorphous state is metastable relative to the crystalline state, transformation of the amorphous material into a crystalline form is possible over time, i.e. during storage, leading to potential decrease in solubility and bioavailability.

A conventional technique for incorporating an amorphous active agent(s) into an oral film dosage form involves dissolving both the amorphous active agent(s)(s) and film forming polymers into a solvent system and quickly evaporating the solvent system and drying the resulting film. The rapid removal of the solvents prevents or at least inhibits nucleation and growth of crystalline forms of the active agent(s). Unfortunately, this technique is not always feasible for various reasons. For example, it is not always possible to find a solvent system in which both the active agent(s) and suitable film forming polymers can be dissolved at sufficiently high concentrations to facilitate rapid evaporation of the solvents and formation of a film containing an adequate concentration of the active agent(s). Further, these dosage forms do not tend to maintain the active agent in a stable amorphous form, but instead the active agent(s) often recrystallize at a sufficiently high rate to prevent practical application of the technique.

Another known technique consists in dry blending an active agent(s) in an amorphous powder form with excipients, optional fillers and optional adjuvants, and then either compressing the blend into a tablet form or disposing the blend in a capsule. However, it is not practicable to use dry blending techniques for incorporating an amorphous powder into a film dosage form when wet casting technique is required.

It is also possible to use a co-extrusion process to incorporate an amorphous powder into a polymer matrix that leaves the extruder in a film form. However, this is only applicable for an active agent(s) having a melting point below or similar to the melting point of the other film components. This can be an expensive technique that imposes high shear stresses on the film forming polymer(s) and the active agent(s), as well as possibly exposing these materials to high temperatures that could induce modifications of the film properties and/or chemical degradation of the active agent(s) and film forming polymer(s).

There remains a need for an improved and/or alternative cost-effective oral film dosage form in which an active agent(s) is incorporated in an amorphous form and remains stable despite processing, manufacturing, and storage constraints.

### SUMMARY OF THE DISCLOSURE

Disclosed is a process for preparing a product containing a stable dispersion of non-solubilized amorphous or partially amorphous particles (powders) of an active agent(s) in an oral film dosage form. The active agent(s) is first fully or partially converted to an amorphous form using any of the various known techniques in the art, including extrusion, solvent evaporation, physical mixture, lyophilization, co-precipitation, nanosuspension, melting, co-melting and spray drying. Next, a liquid film-forming formulation including at least one film-forming polymer and a solvent system is prepared independently. The amorphous or partially amorphous active agent(s) is then suspended in the liquid film-forming formulation without solubilizing the amorphous or partially amorphous active agent(s). When a uniform suspended mixture is obtained, the solvent system is removed, such as by evaporation in a drying oven, to form an oral film dosage form in which the active agent(s) is uniformly distributed in the fully or partially amorphous form. The dimensions of the solid particles within the oral film can be below a certain size range in order to avoid an unacceptable rough feeling while touching the product and during oral ingestion. It was established that a mean particle size of D50 equal or below 250 μm will not create any roughness and/or unpleasant granular feeling. The stability of the finished product is assessed through rate (profile) and recovery using dissolution technique.

In certain aspects of this disclosure, the amorphous form of the active agent(s) dissolves in water at a faster rate than a crystalline form of the active agent(s).

In certain aspects of this disclosure, the amorphous form of the active agent(s) is more soluble in water than a crystalline form of the active agent(s).

In certain aspects of this disclosure, the active agent can also be partially crystalline.

In certain other aspects of this disclosure, the process includes a step of dispersing the active agent(s) with a polymer dispersant to produce an active agent(s) polymer-based particle system that maintains the active agent(s) in the metastable amorphous form and sustains supersaturation of the active agent(s), thereby preventing or retarding crystal nucleation and crystal growth.

These and other features, advantages and objects of the various embodiments will be better understood with reference to the following specification and claims.

### DETAILED DESCRIPTION

The disclosed product is an oral thin film dosage form of a stable dispersion of non-solubilized amorphous or partially amorphous active agent(s), having a mean particle size diameter D50 equal or lower than 250 μm, that remains uniformly distributed within the film with good physical characteristics (e.g., flexibility, dimensions, disintegration) and exhibits enhanced dissolution rate as compared with a crystalline form of the same active agent(s).

The product disclosed herein generally involves preparing, obtaining, or otherwise providing an active agent(s) in an amorphous form or partially amorphous form, optionally combining the active agent(s) with a polymer dispersant to obtain an active agent(s) polymer-based particle system referred as a solid dispersion, uniformly suspending the amorphous active agent(s) in a liquid film-forming formulation, and removing solvents (such as by evaporation in a drying oven) to obtain a solid oral film dosage form that contains the amorphous or partially amorphous active agent(s) uniformly and stably distributed in a polymer matrix.

An “oral film dosage form” generally refers to an edible composition that can be ingested by a subject (human or animal) to orally administer a predetermined amount of an active agent(s) to the subject, wherein the composition is in the form of a film.

The term “film” refers to a type of dosage form that is distinctly different from pills, tablets, caplets, and capsules, and in which the dosage form is a thin strip of material. Such films are typically rapidly disintegrating or rapidly dissolving, but can also exhibit longer disintegration time when required. The films are generally sufficiently flexible to allow bending or even folding without breaking. The films typically have length and width dimensions on the order of 5 to 25 mm, although larger or smaller dimensions are possible and may be desirable in particular circumstances, and a thickness on the order of 5 to 200 μm, although larger or smaller thicknesses are possible and may be desirable in certain circumstances.

The term “active agent(s)” refers mainly to pharmaceutically active ingredients, but may also refer to generally any agent(s) that chemically interacts with the subject to which it is administered to cause a biological change, such as, but not limited to eliminating symptoms of disease or regulating biological functions.

The term “amorphous” refers to the non-crystalline form of the solid, a state that lacks the regular crystalline organization of atoms. The amorphous content (amorphicity) of a solid can be accurately and precisely assessed using a number of well-established methodologies, including isothermal calorimetry, Powder X-ray diffraction (PXRD), Differential Scanning calorimetry (DSC), Continuous Relative Humidity Perfusion Microcalorimetry (cRHp), and Dynamic Vapor Sorption (DVS). In this document, the term amorphous also refers to an active agent(s) that exhibits 30% or more than 30% of amorphous material, more preferably above 50%.

The term “stable” refers to a product which exhibit no changes in the dissolution profile and recovery when the product is exposed to normal stability conditions (example 25° C./60%RH and 40° C./75%RH) for extended period of time.

Examples of drugs that could be beneficially employed in the disclosed processes include aceclofenac, adenosine, adriamycin, alfacalcidol, alosetron, alprazolam, amoxacilline, amphetamine sulfate, aripiprazole, aspirin, atorvastatin calcium, atropine, bacitracin, bicalutamide, bosentan, budesonide, buspirone, carbamazepine, celecoxib, cilostazol, cisapride, citalopram, clofazimine, clopidogrel bisulfate, cyclosporin, cyproterone acetate, delta-9-tetrahydrocannabinol, danazol, delavirdine, desloratadine, dexamethasone, diazepam, diclofenac, dipyridamole, docetaxel, dolargin, domperidine, domperidone, donepezil, doxorubicin, efavirez, entacapone, estazolam, everolimus, ezetimibe, felodipine, flunitrazepam, flutamide, folic acid, fulvestran, furosemide gefitinib, gliperizide, griseofulvin, hydrocortisone, ibuprofen, indomethacin, itraconazone, ketoconazole, ketoprofen, landoprazole, lenalidomide, levonorgestrel, loperamide, loratadine, lovastatin, lysozyme, mecamylamine, metaphetamine, morphine, naproxen, naproxone, nifedipine, nitrazepam, norethindrone, norgestimate, norgestrel, ofloxacin, olanzepine, omeprazol, paclitaxel phytosterol, pimozide, piroxicam, prazepam, progesterone, raloxifene HCl, raloxifene, ridogrel, salicylic acid, simvastatin, stigmasterol, tadalafil, temsirolimus, terfenadine, tolvaptam, tracolimus, triclabendazole, trypsinsulin, tubocurarine, zidovudine ziprazidone, and β-Estradiol.

The term “non-solubilized” means that the majority of the amorphous or partially amorphous active agent(s) is uniformly distributed as a solid particle into a polymer matrix (e.g., a continuous and homogenous solid phase). The particle needs to be non-solubilized in order to maintain the stability of the film product. Solubilizing the amorphous active agent(s) could lead to potential recrystallization of the active agent over time, which may adversely affect the overall bioavailability of the product.

The mean particle size diameter D50 equal or lower than 250 um, refers to the size distribution of the solid particle uniformly distributed in the matrix film. The size can be small enough to avoid any roughness on the texture or bad mouth feel experience when orally ingested.

Liquid film-forming formulations generally include at least one film-forming polymer and a solvent system. The solvent system can be comprised of a single solvent or a mixture of two or more solvents that are typically miscible. The liquid film-forming formulation may incorporate other ingredients that enhance or modify the functionality, processibility, taste or aesthetics of the film. Such film-forming additives include colorants, opacifiers, flavorants, plasticizers, surfactants, etc.

Water soluble polymers that can be employed in the disclosed films include water soluble cellulose derivatives, including hydroxypropylmethyl cellulose, hydroxyethyl cellulose, hydroxypropyl cellulose, polyvinyl pyrrolidone, copovidone (a copolymer of 1-vinyl-2-pyrrolidone and vinyl acetate), other copolymers of vinyl pyrrolidone, other polymers or copolymers of substituted vinyl pyrrolidone, derivatives of polyvinyl pyrrolidone, polyethylene oxide (PEO), carboxymethyl cellulose, polyvinyl alcohol, polysaccharides, natural gums, including xanthan, tragacanth, guar, acacia and arabic gums, and water soluble polyacrylates. As well, practically water insoluble polymers, such as microcrystalline cellulose, ethyl cellulose, and hypromellose phthalate can be used in the formulation. Combinations of these water soluble and non-water soluble polymers or other polymers can also be used.

In certain aspects of this disclosure, a process of making a product as disclosed includes a step of dispersing the active agent(s) in a dispersant to stabilize the amorphous form of the active agent(s) by inhibiting crystal nucleation and crystal growth. A solid dispersion powder, comprising the active agent(s) in an amorphous form and a polymer dispersant, can be produced using known techniques. Examples of polymers that can be used as dispersants include cellulose acetate, cellulose acetate phthalate, copovidone, ethylcellulose, eudragit E, eudragit NE, eudragit L & S, eudragit RL & RS, hydroxypropyl cellulose, hypromellose, hypromellose phthalate, hypromellose succinyl acetate, ethylene glycol, propylene glycol block copolymers (polaxamer), polyethylene glycol, polymethacrylates, polyvinyl acetate phthalate, polyvinyl caprolactam-polyvinyl acetate-polyethylene glycol graft copolymer and povidone.

The amorphous active agent(s) or solid dispersion containing the amorphous active agent(s) is mixed with the liquid film-forming formulation without dissolving the active agent(s).

The term “suspended” (and variations thereof) refers to a dispersion of solid material (e.g., particles or powder) in a bulk liquid medium, in which the solid material is not completely dissolved on a molecular level, and will eventually settle out of the liquid in the absence of agitation. In a suspension, the suspended material is not dissolved in the liquid.

The suspension is mixed at a certain speed and for a limited time to produce a blend that has a uniformity of content of the suspended active agent(s), an acceptable low level of air bubbles, and maintains the amorphous nature of the active agent(s). In addition, to further strengthen the stability of the produced film, dispersant with known stability effect on recrystallization prevention can be used. Stability of the oral film includes stability against the formation of degradation products over a defined period of time, as well as maintained amorphicity and resistance to heat. The stability of the oral film can also be assessed by the dissolution profile of the active agent(s). Any decrease in the rate of dissolution is an indicator of amorphicity change of the active agent(s) within the film product.

The mixing speed in the wet blend is sufficient to introduce the amorphous or partially amorphous active agent(s) and create a film suspension with uniformity of content as per definition in the United States Pharmacopeia (USP) test 905. The mixing time is the time required in combination with the mixing speed to create a uniform suspended mixture that does not solubilize the active agent(s). If the amorphous active agent(s) dissolves in the liquid blend, the physical and chemical characteristics of the final product would change significantly and render the process unstable and unpredictable and increase the chance of potential recrystallization. The wet blend mixing and composition must be set in a way to prevent reagglomeration of the amorphous active agent(s).

The disclosed dosage form will be illustrated by the following non-limiting Example.

### EXAMPLE 1

Using a rotary evaporator, olanzapine and sodium starch glycolate 1:1 are dissolved in acetone. Once fully dissolved, the solvent is removed at 40° C. under reduced pressure until a dry powder is obtained. The powder is collected from the flask and milled until D50 of 75 μm is obtained. This powder is then tested for active pharmaceutical agent(s) assay, particle size distribution, DSC and residual solvent concentration.

A polymer wet blend is created by adding PEO having 100,000 molecular mass and hydroxypropylmethylcellulose (HPMC) having a viscosity of about 50 cP (e.g., 40-60 cP), as measured with Ubbelohde viscometers at a 2% concentration in water at 20° C. (68° F.), (e.g., “Methocel E50”) in a 4:1 mass-ratio pre-mixed together in water containing sucralose under stirring. The blend is mixed under vacuum for at least 3 hours or until a homogenous solution is obtained. The blend is degassed at low speed overnight.

The dry active powder and the wet blend are mixed together using low frequency mixing during a defined period of time and casted right away into a thin film layer. The film layer is dried using an oven with temperature set at a gradient of 50 to 90° C. The produced film sheet is then cut to specific size and individually packaged.

An oral dosage form comprised of amorphous tadalafil is prepared as outlined above and thereafter exposed to a temperature of 40° C. and a relative humidity of 75%. After an extended period of time (e.g., 6 months), it is determined that there is no or very little change in the dissolution profile (i.e., recovery to a deviation of less than 10% from the original dissolution profile) when the oral dosage form is returned to normal conditions (e.g., 25° C. and 60% relative humidity), indicating that the active agent has mostly (e.g., greater than 90%) remained amorphous. Deviation from the original dissolution profile refers to a maximum deviation in the amount of active agent dissolved at any particular time for a dosage form that is exposed to adverse conditions for an extended period of time as compared with a freshly prepared dosage form.

The above description is considered that of the preferred embodiment(s) only. Modifications of these embodiments will occur to those skilled in the art and to those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1. A process for preparing an oral film dosage form containing an active agent in an amorphous form, comprising:
providing the active agent in an amorphous particle form having a mean particle size diameter D50 equal or less than 250 μM;
providing a liquid film-forming formulation including at least one film-forming polymer and a solvent system including at least one solvent, and optionally including one or more pharmaceutically acceptable excipients selected from diluents, plasticizers, surfactants, sweeteners and taste-masking agents;
suspending the active agent in the amorphous particle form in the liquid film-forming formulation without dissolving the active agent; and
removing the solvent system to form a film containing the active agent in the amorphous form and retaining a mean particle size diameter D50 equal or less than 250 μm.

2. The process of claim 1, in which the active agent is provided in the amorphous form by converting a crystalline form of the active agent into the amorphous form using a technique selected from extrusion, solvent evaporation, physical mixture, nanosuspension, melting, lyophilization, co-precipitation, co-melting and spray drying.

3. The process of claim 1, in which the amorphous form of the active agent dissolves in water at a faster rate than a crystalline form of the active agent.

4. The process of claim 1, in which the amorphous form of the active agent is more soluble in water than a crystalline form of the active agent.

5. The process of claim 1, in which the active agent in an amorphous form is selected from the group consisting of aceclofenac, adenosine, adriamycin, alfacalcidol, alosetron, alprazolam, amoxacilline, amphetamine sulfate, aripiprazole, aspirin, atorvastatin calcium, atropine, bacitracin, bicalutamide, bosentan, budesonide, buspirone, carbamazepine, celecoxib, cilostazol, cisapride, citalopram, clofazimine, clopidogrel bisulfate, cyclosporin, cyproterone acetate, delta-9-tetrahydrocannabinol, danazol, delavirdine, desloratadine, dexamethasone, diazepam, diclofenac, dipyridamole, docetaxel, dolargin, domperidine, domperidone, donepezil, doxorubicin, efavirez, entacapone, estazolam, everolimus, ezetimibe, felodipine, flunitrazepam, flutamide, folic acid, fulvestran, furosemide gefitinib, gliperizide, griseofulvin, hydrocortisone, ibuprofen, indomethacin, itraconazone, ketoconazole, ketoprofen, landoprazole, lenalidomide, levonorgestrel, loperamide, loratadine, lovastatin, lysozyme, mecamylamine, metaphetamine, morphine, naproxen, naproxone, nifedipine, nitrazepam, norethindrone, norgestimate, norgestrel, ofloxacin, olanzepine, omeprazol, paclitaxelb phytosterol, pimozide, piroxicam, prazepam, progesterone, raloxifene HCl, raloxifene, ridogrel, salicylic acid, simvastatin, stigmasterol, tadalafil, temsirolimus, terfenadine, tolvaptam, tracolimus, triclabendazole, trypsinsulin, tubocurarine, zidovudine ziprazidone, and β-Estradiol.

6. The process of claim 1, in which the active agent is olanzapine.

7. The process of claim 1, in which the active agent is tadalafil.

8. The process of claim 1, further comprising dispersing the amorphous active agent in a polymer dispersant to stabilize the active agent in the amorphous form by inhibiting crystal nucleation and crystal growth.

9. The process of claim 8, in which the polymer dispersant is selected from the group consisting of cellulose acetate, cellulose acetate phthalate, copovidone, ethylcellulose, eudragit E, eudragit NE, eudragit L & S, eudragit RL & RS, hydroxypropyl cellulose, hypromellose, hypromellose phthalate, hypromellose succinyl acetate, polaxamer, polyethylene glycol, ethylene glycol-propylene glycol block copolymers, polymethacrylates, polyvinyl acetate phthalate, polyvinyl caprolactam-polyvinyl acetate-polyethylene glycol graft copolymer, and povidone.

10. The process of claim 1, in which the film-forming polymer is selected from hydroxypropylmethyl cellulose, hydroxyethyl cellulose, hydroxypropyl cellulose, polyvinyl pyrrolidone, copovidone (copolymer of 1-vinyl-2-pyrrolidone and vinyl acetate), polyethylene oxide, carboxy methyl cellulose, polyvinyl alcohol, polysaccharides, natural gums, water soluble polyacrylates, and combinations of these film-forming polymers.
